(19) United States
(12) Patent Application Publication
(10) Pub. No.: US 2007/0051904 A1
(43) Pub. Date: Mar. 8, 2007

(54) Gantry system for particle therapy, therapy plan or radiation method for particle therapy with such a gantry system

(76) Inventors: Werner Kaiser, Langquaid (DE); Heiko Rohdjess, Grossenseebach (DE); Andres Sommer, Langensendelbach (DE); Tim Use, Nurnberg (DE)

Correspondence Address: BRINKS HOFER GILSON & LIONE, P.O. BOX 10395, CHICAGO, IL, 60610, US

(21) Appl. No.: 11/510,316

(22) Filed: Aug. 25, 2006

Related U.S. Application Data
(60) Provisional application No. 60/712,653, filed on Aug. 30, 2005.

(30) Foreign Application Priority Data
Aug. 30, 2005 (DE) DE 102005041122.3

(51) Int. Cl. G21G 5/00; A61N 5/00

(52) U.S. Cl. 250/492.1

(57) ABSTRACT

A gantry system for particle therapy system, therapy plan and radiation method for a particle therapy with such a gantry system is provided. The gantry system for a particle therapy system includes a particle beam. A first beam guiding device rotatable about an axis of rotation of the gantry guides the particle beam so that the particle beam strikes a treatment location at a gantry incidence direction. The gantry incidence direction deviates from the axis of rotation and is variable. A second beam guiding device guides the particle beam to a treatment location in a second direction.

The present patent document claims the benefit of the filing date under 35 U.S.C. § 119(e) of Provisional U.S. Patent Application Ser. No. 60/712,653 filed on Aug. 30, 2005, which is hereby incorporated by reference. This application also claims the benefit of DE 10 2005 041 122.3, filed Aug. 30, 2005.

### BACKGROUND

1. Field

The present embodiments relate to a gantry system for a particle therapy system, therapy plan and radiation method for a particle therapy with such a gantry system.

2. Related Art

A particle therapy system generally has a particle accelerator unit with a high-energy-beam guiding system. The acceleration of particles, such as protons, pions, helium ions, carbon ions and oxygen ions is completed with the aid of a synchrotron or cyclotron.

The high-energy-beam guiding system guides the particles from the accelerator unit to at least one treatment chamber. The difference between a “fixed beam” treatment chamber and a “gantry-based” treatment chamber is that a “fixed beam” treatment chamber has particles that strike a treatment location from a fixed direction, and particles from the “gantry-based” treatment chamber are provided to a treatment location from different directions.

Gantry devices for particle therapy are heavy, large in size and expensive to manufacture because of the mechanical requirements of at least the magnetic rigidity of the particle beam. For example, a gantry device that uses a carbon ion beam, in comparison to a proton gantry, has considerably larger dimensions and has the same penetration depth into the patient. Thus, the gantry device that uses a carbon ion beam is more complex and more expensive to produce. Proton gantries are generally used in particle therapy systems. For treatment with heavy ions, only medical chambers with a fixed beam angle (fixed-beam treatment chamber) are used, because fixed-beam treatment chamber is less expensive. A disadvantage of the less-expensive fixed-beam treatment chamber is that the patient must be oriented relative to the beam, because the beam can not be oriented relative to the patient.

Typically, a control and safety system of the particle therapy system assures that a particle beam, which has the desired parameters, is guided into the appropriate treatment chamber. The parameters are defined by the treatment or therapy plan desired. This therapy plan indicates how many particles are supposed to strike the patient, and from which direction and with what energy the particles are supposed to strike the patient. The energy of the particles determines the penetration depth of the particles into the patient. For example, particle therapy takes place at the site with the maximum interaction with the tissue. In other words, particle therapy takes place at the site where the maximum dose is deposited to the tissue. During the treatment of a tumor, the maximum deposited dose is located inside the tumor (or in the case of other medical applications of the particle beam, in the applicable target area). The control and safety system furthermore controls a positioning device, with which the patient is positioned relative to the particle beam.

A particle therapy system with a plurality of fixed-beam treatment locations and a gantry is disclosed in European Patent Disclosure EP 0 986 070. Various radiation methods exist, for example, scanning or scattering methods. A scanning method is generally a radiation method that has a particle beam with a transverse extent that is less than the cross-sectional area of the volume to be irradiated. In the conventional radiation method, the particle beam moves across this cross-sectional area and delivers the planned radiation dose to each volume element of the cross-sectional area. Various principles are known in this connection, such as raster scanning or spot scanning. A summary of various treatment systems and techniques is provided for instance by H. Blattman in “Beam delivery systems for charged particles”, Radiat. Environ. Biophys. (1992) 31:219-231.

European Patent Disclosure EP 1 148 911 B1 discloses a gantry system that adjusts and aligns an ion beam with a target. Alternatively, an outfit for radiation with charged particles that reduces overall expenses and allows a particle beam to be aimed from various directions at a target is disclosed in United States Publication No. 2002/0030164. However, one set of beam-shaping elements is used for all the beam incidence directions.

Accordingly, a more flexible system for particle therapy that enables versatile use and efficient utilization of a gantry system of a particle therapy system is desired.

### SUMMARY

In one embodiment of the gantry system, the system has a second beam guiding device. The second beam guiding device diverts the particle beam to the treatment location from an incidence direction that cannot be treated with the first beam guiding device. By means of a launching unit, a choice can be made in the radiation between the two beam guiding devices. In one exemplary embodiment, the gantry system combines the treatment of the patient with heavy particles from a fixed direction in space with lightweight particles (for example, protons) from the gantry incidence directions. The transportation of the patient from a gantry-based medical room to a fixed-beam medical room may be avoided. Thus, radiation of the patient is more efficient, and the additional possible gantry angles of incidence can be accounted for by various particles used. In this embodiment, the patient is first radiated with one type of particle and then radiated with another type of particle, from different incidence directions.

According to one exemplary embodiment, a treatment location is more efficiently utilized because of the combination of fixed-beam and gantry incidence directions, for example, when only a few patients require treatment solely with gantry angles of incidence.

The gantry system of one exemplary embodiment makes it easier to adapt treatment strategies, which allows the mixture of proton treatments with heavier particle treatments. Accordingly, the medical rooms with a proton gantry combined with an integrated fixed beam are not reserved exclusively for proton treatments or heavy particle treatments.

In one embodiment, the second beam guiding device extends in the direction of the axis of rotation of the first beam guiding device. This embodiment may provide for operation with heavy ions, for example, helium ions, carbon ions or oxygen ions. Accordingly, a fixed beam is available in a treatment location based on a gantry and a treatment chamber.

In another exemplary embodiment, a beam guiding shunt that rotates with the gantry is installed on the proton gantry as a switch magnet between the two beam guiding devices. For example, the beam guiding shunt directs the beam guidance either to a part of the gantry system that rotates with the gantry or to a part of the gantry system that extends directly along the axis of rotation. The two beam guiding devices have their own scanning magnets, passive scattering systems and/or beam diagnosis elements. In this exemplary embodiment, the second beam guiding device, the beam diagnosis elements and the other beam-shaping components, for example, quadrupole magnets, are rotated with the gantry. Alternatively, only selected components and devices are rotated with the gantry.

To simplify the use of the beam diagnosis elements, that is, to define a replicable coordinate system with regard to beam diagnosis (beam positioning detection) and patient positioning, the radiation of a patient with particles from the second beam guiding device is done at the same gantry rotation angle. For example, radiation is completed at, but not limited to, the zero-degree position, which is the position where the outlet opening of the rotatable beam guiding device is located above the patient.

In another exemplary embodiment, a quasi-fixed second beam guiding device is rotatable relative to the first beam guiding device. When the first beam guiding device rotates, the second beam guiding device remains unmoved relative to the patient. Accordingly, radiation via the second beam guiding device can be completed at every gantry rotation angle, because the beam-shaping components of the second beam guiding device are triggered independently of their rotation. Thus, the beam parameters remain essentially constant at all positions of the gantry. In this embodiment, the radiation is completed with a fixed beam even while the gantry is moving to a new position, which may save time and may increase precision of the radiation if the patient is not moved.

In an alternative embodiment, the particle therapy system utilizes raster scanning and/or scattering radiation.

The embodiments are not limited to two beam diagnosis systems that determine the position and intensity of the particle beam. Alternatively, a one beam diagnosis system is utilized, which is moved by, for example, a guide or a robot, to the beam exit points of the two beam guiding devices.

### BRIEF DESCRIPTION OF THE DRAWINGS

FIG. 1 illustrates an exemplary embodiment of a gantry system with a second beam guiding device that rotates with the first beam guiding device;

FIG. 2 illustrates an exemplary embodiment of a gantry system with a second guiding device that does not rotate with the first beam guiding device;

FIG. 3 illustrates an exemplary embodiment of a gantry system with a second beam guiding device rotatable relative to the first beam guiding device; and

FIG. 4 is a flow chart that illustrates the course of operation of a particle therapy system with a gantry system according to an exemplary embodiment.

### DETAILED DESCRIPTION

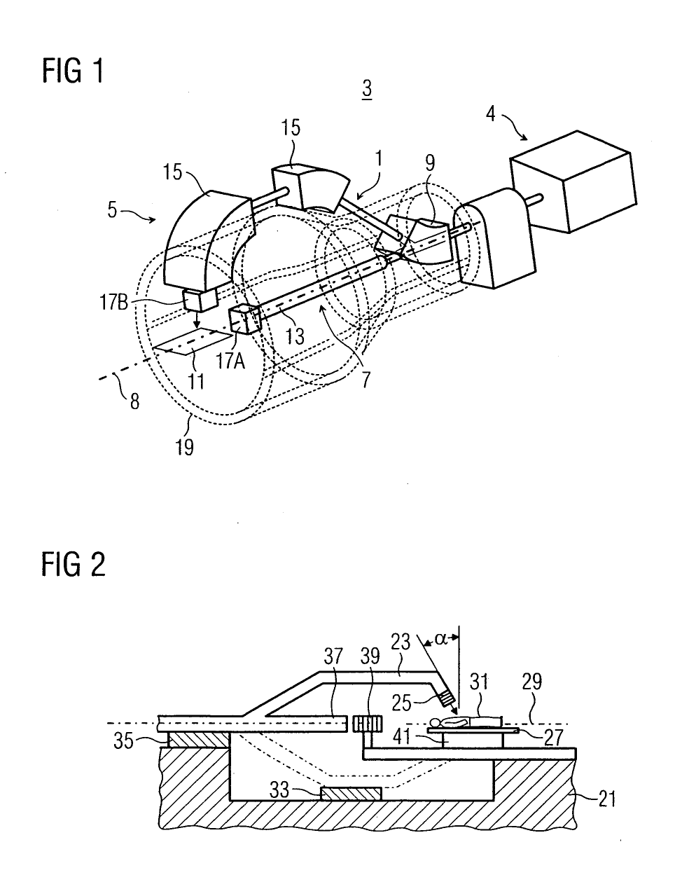

FIG. 1 schematically shows a gantry system 1. A particle beam of a particle therapy system 3 is input into the gantry system 1. The particle therapy system 3 includes an accelerator and beam delivery unit 4. The gantry system 1 includes a first beam guiding device 5 and a second beam guiding device 7. The gantry system 1 can be rotated in at least one angular range about an axis 8 of rotation. A launching unit 9 has a switch magnet coupled to and actuated by a therapy control center of the particle therapy system 3 in accordance with a treatment plan. Depending on the treatment plan entered into the particle therapy system 3, the particle beam strikes a treatment location 11 either at an angle to the axis of rotation 8 or along the axis of rotation 8. In FIG. 1, the beam path 13 of the second beam guiding device 7 extends as a central beam along the axis of rotation 8. In this exemplary embodiment, the beam path 13 is utilized as, but not limited to, a fixed beam. In an alternative embodiment, the beam path 13 is oriented at a small angle and/or slightly offset.

In one exemplary embodiment, the second beam guiding device 7 includes beam-shaping magnets (not shown), for example, quadrupole or dipole magnets. Deflection magnets 15, for example, are utilized with the first beam guiding device 5. Two beam diagnosis systems 17A and 17B furnish information about, for example, the beam location, beam shape, and/or beam intensity. As shown in FIG. 1, the beam diagnosis systems 17A and 17B rotate with the gantry system. In an alternate embodiment, one beam diagnosis system is utilized that is alternatively movable between the two beam exits.

As shown in FIG. 1, the components are mechanically mounted on one gantry mounting structure 19. The gantry mounting structure 19 is moveable in its angular position via one or more bearings (not shown).

FIG. 2 illustrates an alternate embodiment of a gantry system. The gantry system according to this exemplary embodiment includes a platform 21. A first beam guiding device 23 has a fixed beam diagnosis system 25. The first beam guiding device 23 guides a particle beam to strike a treatment location 27 at a gantry angle of incidence that deviates from a direction defined by an axis 29 of rotation. As shown in FIG. 2, the particle beam strikes the patient 31 in a direction deflected from a vertical axis by an angle α. The vertical axis is an axis perpendicular to the axis 29 of rotation.

The gantry system is supported rotatably by two bearings 33 and 35. The second beam guiding device 37 is mechanically coupled to the first beam guiding device 23 and to a gantry mounting structure. In the exemplary embodiment shown in FIG. 2, a beam diagnosis system 39 is not rotatable with the gantry system and is mechanically coupled rigidly to the treatment chamber platform 21. For example, the beam diagnosis system 39 is fixed to the platform 21 via a lengthened base that protrudes into the gantry system. A positioning device 41 can additionally be located on the lengthened base.

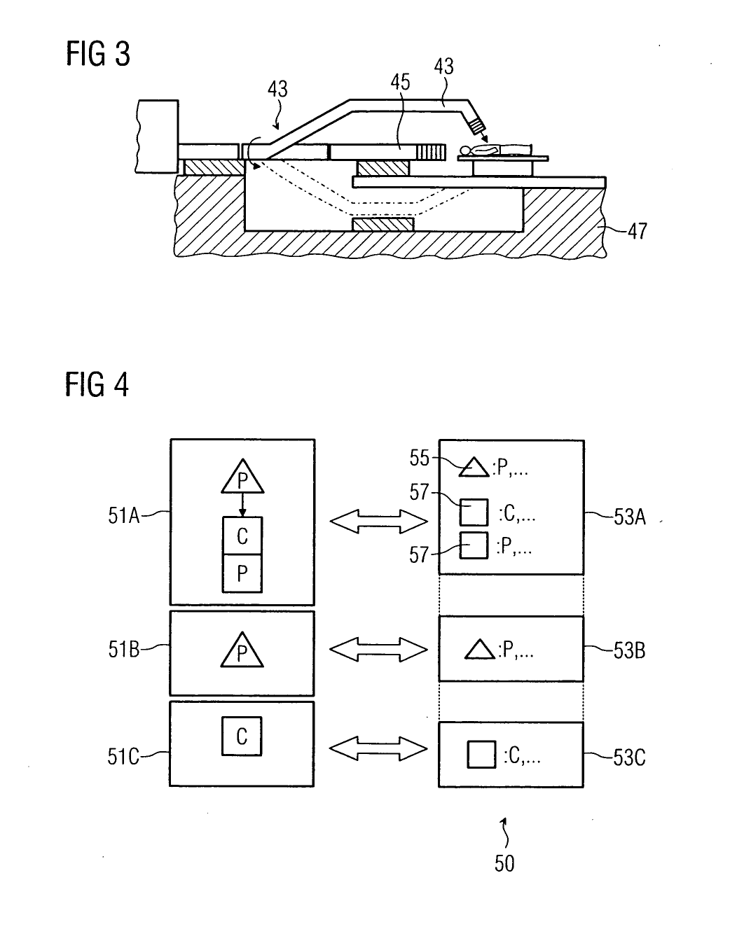

FIG. 3 illustrates an alternative embodiment of a gantry system that includes a first beam guiding device 43 that is similar to the embodiment shown. in FIG. 2. The embodiment shown in FIG. 3 is different from the gantry systems of FIGS. 1 and 2 in that the second beam guiding device 45 has a rotatable coupling. The second beam guiding device 45 is mechanically uncoupled from a platform 47. According to this embodiment, beam-shaping elements may stay in a launching unit 49 embodied as a switch magnet.

This embodiment of the gantry system simplifies radiation with the second beam guiding device 45. The beam guiding device is independent of the angular position of the first beam guiding device 43. Accordingly, no correction or only a slight correction of the beam geometry has to be made in the second beam guiding device 45.

FIG. 4 is a flow chart that illustrates one example of a course of treatment plan 50 using gantry systems that have a first and a second beam guiding device. In the treatment plan 50, the types of particles to be used, for example, for the radiation, the radiation intensity, the particle energy, and the beam intensity are defined.

The treatment blocks 51A, 51B, and 51C represent one radiation session for one patient. Each radiation session is based on a partial treatment plan 53A, 53B and 53C. The partial treatment plans include, for example, radiation procedures 55 that utilize a first beam guiding device, radiation procedures 57 that utilize a second beam guiding device, or any combination thereof

In the treatment block 51A, a patient is treated both via the first beam guiding device and via the second beam guiding device. The second beam guiding device is used both for treatment with carbon and for treatment with protons. In treatment block 51B, a patient is treated with radiation via the first beam guiding device, which is operated with protons. The treatment plans are not limited to the specific particles disclosed, for example, other suitable particles that are known or will be known in the art may be utilized.

In treatment block 51C, the option of being able to use the gantry system as a gantry is not needed. The patient is treated with radiation that utilizes a carbon ion beam that is not adjustable in direction. The radiation of this exemplary embodiment is not limited to a carbon ion beam, other suitable particle beams that are known or will be know in the art may be utilized. In the present exemplary embodiment, the treating plans have increased flexibility, for example, the treatment plans used to treat different patients that are treated in succession may be advantageously selected.

Various embodiments described herein can be used alone or in combination with one another. The forgoing detailed description has described only a few of the many possible implementations of the present invention. For this reason, this detailed description is intended by way of illustration, and not by way of limitation. It is only the following claims, including all equivalents that are intended to define the scope of this invention.

What is claimed is:

1. A gantry system for a particle therapy system for irradiating a volume, the system comprising:
a first beam guiding device rotatable about an axis of rotation of a gantry, wherein the first beam guiding device is operable to guide the particle beam so that the particle beam strikes a treatment location at a gantry incidence direction, and wherein the gantry incidence direction deviates from an axis of rotation and is variable; and
a second beam guiding device is operable to guide the particle beam to a treatment location in a second direction different than the gantry incidence direction.

2. The gantry system as defined by claim 1, wherein the second direction is parallel to the axis of rotation.

3. The gantry system as defined by claim 1, wherein the second beam guiding device is non-rotatable and is mechanically uncoupled from the first beam guiding device.

4. The gantry system as defined by claim 1, wherein the second beam guiding device is mechanically coupled to the first beam guiding device and is operable to rotate with the first beam guiding device.

5. The gantry system as defined by claim 1, further comprising a beam monitoring unit that monitors particle beam parameters.

6. The gantry system as defined by claim 5, wherein the beam monitoring unit is disposed on the first beam guiding device or the second beam guiding device.

7. The gantry system as defined by claim 5, wherein the beam monitoring unit is mechanically independent of the first beam guiding device and the second beam guiding device.

8. The gantry system as defined by claim 1, wherein the first beam guiding device or the second beam guiding device has a scanner, scattering device, or both.

9. The gantry system as defined by claim 1, wherein the first beam guiding device is operable for protons and the second beam guiding device is operable for protons or heavy ions.

10. A particle therapy system comprising: a gantry system operable for a particle beam; a first beam guiding device rotatable about an axis of rotation of the gantry, wherein the first beam guiding device is operable to guide the particle beam so that the particle beam strikes a treatment location at a gantry incidence direction, and wherein the gantry incidence direction deviates from the axis of rotation and is variable; and a second beam guiding device operable to guide the particle beam to a treatment location in a second direction.

11. The particle therapy system as defined by claim 10, wherein the particle therapy system is operable with at least two types of particles.

12. An operating method for operating a particle therapy system having a gantry system, the method comprising:
radiating in a first gantry incidence direction that is adjustable by rotation of a first beam guiding device, and
radiating in a second, different, incidence direction with a second beam guiding device.

13. A method for a treatment plan for irradiating a patient with particles of a particle therapy system having a gantry system, the method comprising:
radiating in a first gantry incidence direction that is adjustable by rotation of a first beam guiding device, and
radiating in a second incidence direction with a second beam guiding device, wherein the plan has a first sub-treatment plan for radiating with the first beam guiding device and a second sub-treatment plan for radiating with the second beam guiding device.

14. The method as defined by claim 13, wherein the first sub-treatment plan is based on the radiation parameters of the particle number, particle energy, particle type, or any combination thereof and on a defined gantry angle of incidence.

15. The gantry system as defined by claim 1, wherein the second beam guiding device is operable for a fixed-beam radiation treatment.

16. The gantry system as defined by claim 5, wherein the beam monitoring unit is operable to monitor beam position.

17. The gantry system as defined by claim 5, wherein the beam monitoring unit is operable to monitor beam intensity.

18. The particle therapy system as defined by claim 10, wherein the particle therapy system is switchable between two or more of the types from the group of: protons, pions, helium ion, carbon ion or oxygen ion therapy.

19. The method as defined by claim 12, further comprising radiating a patient from either the first gantry incidence direction or the second incidence direction.

20. The method as defined by claim 12, further comprising radiating a first patient from the second incidence direction being a random incidence direction deviating from possible gantry angles of incidence.

21. The treatment plan as defined by claim 13, wherein the second sub-treatment plan defines a radiation based on the radiation parameters of the particle number, particle energy, particle type, or any combination thereof.

22. The gantry system as defined by claim 1, wherein the second direction is a direction not obtainable by the first beam guiding device.
